(19) United States
(12) Patent Application Publication
(10) Pub. No.: US 2005/0020495 A1
(43) Pub. Date: Jan. 27, 2005

(54) Antiretroviral compositions comprising thymosin alpha peptides and protease inhibitors

(76) Inventors: Eduardo Bruno Guimaraes Martins, Foster City, CA (US); Nicholas Paton, Singapore (SG)

Correspondence Address: ROTHWELL, FIGG, ERNST & MANBECK, P.C., 1425 K STREET, N.W., SUITE 800, WASHINGTON, DC, 20005, US

(21) Appl. No.: 10/492,189

(22) PCT Filed: Oct. 23, 2002
(86) PCT No.: PCT/US02/33802

Related U.S. Application Data
(60) Provisional application No. 60/330,549, filed on Oct. 24, 2001.

(51) Int. Cl. A61K 38/17; A61K 31/551; A61K 31/522

(52) U.S. Cl. 514/12; 514/220; 514/263.31

(57) ABSTRACT

The present invention provides a method and a pharmaceutical combination for treating a human infected with HIV utilizing an HIV, treatment-effective amount of a Tα1 peptide, and an HIV, inhibitarialy-effective amount of at least one pratease inhibitor compound.

### BACKGROUND OF THE INVENTION

1. Field of the Invention

The present invention relates to the treatment of HIV.

2. Description of the Background Art

Human immunodeficiency virus (HIV) attacks helper T cells, the component of the immune system that is integral to fighting infections. Due to the weakening of the immune system, infected individuals are susceptible to opportunistic infections. Due to its highly variable and mutable coat characteristics, HIV infection is difficult to treat especially using traditional vaccine treatment. Sine no anti-HIV vaccine is currently available, research has focused on treatment of the disease. Current treatment involves the use of antiviral therapy in order to target reverse trancriptase and halt viral replication.

Treatment of patients infected with HIV with highly-active antiretroviral therapy (HAART) is associated with immune reconstitution as evidenced by increased absolute CD4 T cells as well as reduced opportunistic infections and improved survival. In a minority of patients however, especially those starting therapy with low CD4 counts, CD4 recovery is incomplete and such individuals appear at greater risk of AIDS-associated events than patients with substantial recovery of CD4 counts. This has prompted the investigation of a number of immunomodulatory compounds with a view to enhancing immune reconstitution with HAART.

Thymosin α1 (Tα1) is a 28-amino acid thymic peptide hormone with immunomodulatory properties, homologous to a natural product originally isolated from thymosin fraction 5 of calf thymuses. Its biological effects include augmentation of T lymphocyte function and include modulation of interleukin-2 (IL-2), stimulation of interferon-γ production, induction of T lymphocyte and NK cell, and stimulation of thymopoiesis. Thymosin al also has been shown to up-regulate MHC Class I expression in antigen-presenting cells.

Although Thymosin al has previously been suggested for use in certain treatments of HIV, there remains a need in the art for improved methods of combination treatment for HIV.

### SUMMARY OF THE INVENTION

The present invention provides a method and a pharmaceutical combination for treating a human infected with HIV comprising an HIV treatment-effective amount of a Tα1 peptide, and an HIV inhibitorialy-effective amount of at least one protease inhibitor compound.

### DETAILED DESCRIPTION OF THE INVENTION

The present invention is based on a discovery that a pharmaceutical combination of an HIV treatment-effective amount of a Tα1 peptide, e.g., an amount of a Thymosin α 1 peptide sufficient to increase T cell receptor circles (TREC) in a patient, and an HIV inhibitorialy-effective amount of at least one protease inhibitor (PI) compound, is effective for treating a human infected with HIV.

Without being bound to any particular theory, it appears that this combination has the capacity to stimulate immune reconstitution which is shown by an increase in TREC circles.

The present invention provides factors and compositions that can enhance immune reconstitution and increase TREC in individuals infected with HIV.

Thymosin α1 has induced increases in CD4 counts when given in combination with either IL-2 or interferon-α (INF-α). In certain clinical trials, thymosin al was administered to patients taking zidovudine monotherapy. One notable result of these studies was the virtual absence of any significant systemic adverse reactions in patients taking thymosin al in contrast to those taking IL-2 or INF-α.

The role of de novo naive T cells produced by thymopoiesis may be an important component of immune reconstitution in patients taking HAART. Until recently, reliable markers of de novo T cells have not been available to gauge thymopoiesis. However, levels of T cell receptor rearrangement circles (TREC) in T lymphocytes appear to correlate with recent thymic emigration and thus thymopoiesis, as well as clinical progression and mortality in HIV-infected individuals. In the presence of potent antiretroviral therapy leading to highly effective suppression of viral replication, thymosin al appears to have greater efficacy in inducing immune reconstitution, particularly through increasing de novo naive T lymphocyte expansion.

In accordance with one embodiment, the invention is a pharmaceutical combination for treating a human infected with HIV, comprising an amount of a Tα1 peptide, preferably Tα1, sufficient to increase TREC, and an HIV inhibitorialy-effective amount of at least one protease inhibitor.

An “inhibitorialy effective amount” of protease inhibitor is an amount of the drug which inhibits HIV replication.

The invention is applicable to Tα1 peptides including naturally occurring Tα1 as well as synthetic Tα1 and recombinant Tα1 having the amino acid sequence of naturally occurring Tα1, amino acid sequences substantially similar thereto, or an abbreviated sequence form thereof, and their biologically active analogs having substituted, deleted, elongated, replaced, or otherwise modified sequences which possess bioactivity substantially similar to that of Tα1, e.g., a Tα1 peptide having sufficient amino acid homology with Tα1 such that it functions in substantially the same way with substantially the same activity as Tα1.

In one embodiment, the invention is a pharmaceutical combination for treating a human infected with HIV, comprising a Tα1 peptide and at least one protease inhibitor, where the dosage of the Tα1 peptide is about 1-10 mg. Preferred dosages which may be used in accordance with the present invention include about 2.2-10 mg Tα1. The amount of Tα1 administered to treat HIV is an amount that is sufficient in conjunction with administration of protease inhibitor to significantly affect a positive clinical response. In preferred embodiments, the amount Tα1 which may be administered is about 3.2 mg.

In another embodiment, the invention is a pharmaceutical combination for treating a human infected with HIV comprising a Tα1 peptide and at least one protease inhibitor where the dosage of the protease inhibitor is about 1-1000 mg. The protease inhibitors which may be used in accordance with the present invention may include, e.g., ritonavir, saquinavir, nelfinavir, indinavir, amprenavir and combinations thereof.

In another embodiment, the invention is a pharmaceutical combination for treating a human infected with HIV comprising an effective amount of a Tα1 peptide, an HIV inhibitorialy-effective amount of at least one protease inhibitor, plus an effective amount of a compound selected from the group consisting of nucleoside reverse transcriptase inhibitors (NRTIs), non-nucleoside reverse transcriptase inhibitors (NNRTIs) and combinations thereof. NRTIs and NNRTIs prevent healthy T-cells in the body from becoming infected with HIV.

The NRTIs which may be used in accordance with the present invention may include AZT, 3TC, ddI, d4T and abacavir.

The NNRTIs which may be used in accordance with the present invention may include efavirenz, nevirapine and delavirdine.

In another embodiment, the invention is a method for treating a human infected with HIV, comprising administering to said person a pharmaceutical combination comprising an effective amount of a Tα1 peptide, and an effective amount of at least one protease inhibitor. Dosages of Tα1 which may be used in accordance with the method are as outlined above. The dosages of protease inhibitor which may be used in accordance with the present also are as outlined above. Suitable protease inhibitor compounds may include ritonavir, saquinavir, nelfinavir, indinavir, amprenavir and combinations thereof.

Administration of the dosages can be intravenous, subcutaneous, intramuscular, or any other acceptable method. Administration of the described dosages can occur for a length of time, effective to reduce or eliminate HIV infection in the patient. Doses are administered preferably over a nine month course of treatment by subcutaneous injection. The amount of drug administered and the treatment regimen used will, of course, be dependent on the age, sex and medical history of the patient being treated, the severity of the specific disease condition and the tolerance of the patient to the treatment as evidenced by local toxicity and by systemic side-effects.

For any route of administration, divided, single or multiple dosage units may be used. Separate dosage units of Tα1 and protease inhibitor can be administered to the patient concurrently over a course of treatment.

In another embodiment, the invention is a method for treating a human infected with HIV, comprising administering to said person a pharmaceutical combination comprising an effective amount of a Tα1, an HIV inhibitorialy-effective amount of at least one protease inhibitor, plus at least one compound selected from the group consisting of nucleoside reverse transcriptase inhibitors (NRTIs), non-nucleoside reverse transcriptase inhibitors (NNRTIs) and combinations thereof.

The NRTIs which may be used in accordance with the present invention may be selected from the group consisting of AZT, 3TC, ddI, d4T and abacavir.

The NNRTIs which may be used in accordance with the present invention may be selected from the group consisting of efavirenz, nevirapine and delavirdine.

In another embodiment, the invention is a method for treating a human infected with HIV, comprising administering to said human Tα1 at a dosage within a range of about 2.2-10 mg, preferably about 3.2 mg.

The invention is further illustrated by the following example, which is not to be construed as limiting.

### EXAMPLE

Treatment of HIV in Human Patients

Efficacy of HIV treatment is shown by evaluating TREC response in peripheral blood mononuclear cells (PBMCs) to treatment with Tα1 plus an HIV inhibitory agent.

Twenty clinically stable patients with viral loads <400 copies/ml and CD4 counts less than 200 cells/μl were randomised to receive 3.2 mg thymosin Tat subcutaneous injections twice weekly or no injections for 12 weeks. CD4 and CD8 counts, CD45 RO+ and RA+ subsets and T cell re-excision circles (TREC) in peripheral blood mononuclear cells (PBMCs) were measured every four weeks, along with regular clinical assessments and routine laboratory tests.

Of the 20 patients, 13 were randomized to receive Tα1 and 7 were controls. Tα1 was well tolerated and no patients taking the medication developed signs of toxicity during the trial. The changes in CD4, CD8 and CD45 lymphocyte subsets did not differ between the groups over the 12 week study period, however there was a significant increase in TREC levels in patients given Tα1 at week 12 compared to controls.

A large proportion of samples failed to yield detectable levels of TREC, most likely due to insufficient quantities of cells available for analysis. Despite this, it was possible to calculate absolute mean values and change in mean values in both study groups using non-parametric tests. The mean TREC levels were similar in each group at each time point except week 12, when far more of the Tα1 group had detectable TREC levels than control patients, and mean levels of TREC were 20 times greater in this group. Furthermore, there was a significant difference between change in TREC levels at week 12 compared to baseline in patients in the Tα1 group compared to control patients.

1. A pharmaceutical combination for treating a human infected with HIV, comprising:
a) an HIV treatment-effective amount of a Thymosin α 1 (Tα1) peptide, and
b) an HIV inhibitorialy-effective amount of at least one protease inhibitor (PI) compound.

2. The pharmaceutical combination of claim 1 wherein the amount of Tα1 peptide is sufficient to increase T cell receptor circles (TREC) in said patient.

3. The pharmaceutical combination of claim 1 wherein the Tα1 peptide is Tα1.

4. The pharmaceutical combination of claim 3 wherein Tα1 is at a dosage of about 1-10 mg.

5. The pharmaceutical combination of claim 4 wherein Tα1 is at a dosage of about 2.2-10 mg.

6. The pharmaceutical combination of claim 5 wherein Tα1 is at a dosage of about 3.2 mg.

7. The pharmaceutical combination of claim 1 wherein said protease inhibitor compound is at a dosage within a range of about 1-1000 mg.

8. The pharmaceutical combination of claim 7 wherein said protease inhibitor compound is selected from the group consisting of ritonavir, saquinavir, nelfinavir, indinavir, amprenavir and combinations thereof.

9. The pharmaceutical combination of claim 1 wherein said combination further comprises an HIV treatment-effective amount of at least one compound selected from the group consisting of nucleoside reverse transcriptase inhibitors (NRTIs), non-nucleoside reverse transcriptase inhibitors (NNRTIs) and combinations thereof.

10. The pharmaceutical combination of claim 9 wherein said NRTIs are selected from the group consisting of AZT, 3TC, ddI, d4T and abacavir, and said NNRTIs are selected from the group consisting of efavirenz, nevirapine and delavirdine.

11. A method for treating a human infected with HIV, comprising administering to said person a pharmaceutical combination as defined in claim 1.

12. The method of claim 11 wherein said Ta1 peptide is Tα1.

13. The method of claim 12 wherein said Tα1 is at a dosage of about 1-10 mg.

14. The method of claim 13 wherein said Tα1 is at a dosage of about 2.2-10 mg.

15. The method of claim 14 wherein said Tα1 is at a dosage of about 3.2 mg.

16. The method of claim 11 wherein said protease inhibitor compound is at a dosage within a range of about 1-1000 mg.

17. The method of claim 16 wherein said protease inhibitor compound is selected from the group consisting of ritonavir, saquinavir, nelfinavir, indinavir, amprenavir and combinations thereof.

18. The method of claim 11 wherein said combination further comprises an HIV treatment-effective amount of at least one compound selected from the group consisting of nucleoside reverse transcriptase inhibitors (NRTIs), non-nucleoside reverse transcriptase inhibitors (NNRTIs) and combinations thereof.

19. The method of claim 18 wherein said NRTIs are selected from the group consisting of AZT, 3TC, ddI, d4T and abacavir, and said NNRTIs are selected from the group consisting of efavirenz, nevirapine and delavirdine.

20. A method for treating a human infected with HIV, comprising administering to said human a Thymosin α1 peptide at a dosage within a range of about 2.2-10 mg.

21. The method of claim 20 wherein said Tα1 peptide is Tα1.

22. The method of claim 21 wherein said dosage is about 3.2 mg.
